(19) United States
(12) Patent Application Publication
(10) Pub. No.: US 2023/0212521 A1
(43) Pub. Date: Jul. 6, 2023

(54) METHOD FOR MASS PROLIFERATION OF URINE-DERIVED PLURIPOTENT CELLS

(71) Applicant: PURICELLMANIA CO., LTD., Seoul (KR)

(72) Inventors: Jong Bin KIM, Seoul (KR); Moon Young KIM, Seoul (KR)

(73) Assignee: PURICELLMANIA CO., LTD., Seoul (KR)

(21) Appl. No.: 18/000,718

(22) PCT Filed: May 25, 2021
(86) PCT No.: PCT/KR2021/006477

§ 371 Date: Dec. 5, 2022

(30) Foreign Application Priority Data
Jun. 5, 2020 (KR) 10-2020-0068444

(51) Int. Cl. C12N 5/071

(52) U.S. Cl. CPC C12N 5/0684; C12N 2501/11; C12N 2501/33; C12N 2501/39; C12N 2501/115; C12N 2501/235; C12N 2501/998; C12N 2501/2302; C12N 2511/00

(57) ABSTRACT

A method for the mass proliferation of urine-derived multipotent cells and a medium composition for the mass proliferation of urine-derived multipotent cells according to the present invention can be used to massively proliferate urine cells by efficiently isolating the same even from urine that has been left alone for a long period of time, and can be used to produce multipotent cells having characteristics of epithelial cells, mesenchymal cells, and stem cells.

### TECHNICAL FIELD

The present invention relates to a method of massively proliferating multipotent cells in urine.

### BACKGROUND ART

Urine is a by-product of blood filtered through the kidneys and waste of the human body, which are discharged out of the body through the bladder and urethra, and since it includes several metabolites in the human body, is used to diagnose diseases including kidney and urinary system diseases, endocrine diseases such as diabetes, metabolic diseases such as hormone abnormalities, and various types of cancer such as prostate cancer, liver cancer, kidney cancer, bladder cancer, etc. In addition, urine has all of physical characteristics such as color, turbidity, odor or pH; chemical characteristics such as minerals, fatty acids or vitamins; and biological characteristics such as genes, proteins or cells, which show various aspects according to human body conditions, and is used as a useful indicator for diagnosing diseases.

In order to diagnose a disease, compared to when blood and tissue are used, when urine is used, a larger amount of test sample can be collected within a short time, and urine collection is not painful in a patient compared to other types of tissue collection, and urine can be not only collected several times by time and date, but also is easy to diagnose and follow up various diseases at the same time using the collected samples.

Cells contained in urine, which is one of the by-products of urine, can also be used to diagnose and treat various diseases. To obtain cells in the human body, the cells have to be directly isolated from a tissue section, or isolated from blood flowing through a blood vessel using a syringe and cultured. A method of acquiring cells using the above-mentioned method causes extreme pain to a patient, is difficult to obtain the cells several times, has to be accomplished by a highly skilled person, and incurs a lot of costs due to various materials required for collection. Particularly, in the human body, epithelial cells have to be isolated and cultured after directly cutting out the skin or tissue in the human body using a surgical knife, and it is difficult to massively proliferate the cells.

If cells with characteristics in human tissue or blood from urine can be isolated and cultured, it is possible to efficiently separate and culture cells at a lower cost without causing pain to a patient. In particular, when multipotent cells with the characteristics of epithelial cells can be isolated and cultured, and proliferated on a large scale, it will be useful for diagnosing and treating many diseases caused by a lack of epithelial cells. However, when urine contains only a very small number of cells and exceeds the pH range maintaining cells in the urine, the cells do not survive for a long time in the urine. In addition, since there are only a small number of cells in the urine, it is difficult to confirm a precipitate even by centrifugation for isolating cells, and it is even more difficult to separate urine cells from the precipitate. That is, it is difficult to obtain urine cells from urine and culture the obtained urine cells, so the cells are not used in diagnosis and treatment of a disease.

With respect to the technology for culturing cells isolated from urine, KR Unexamined Patent Application Publication No. 10-2017-0126564 discloses a medium composition for stable culture of urine-derived cells having the culture characteristics of renal progenitor cells under the premise that urine has the characteristics of renal progenitor cells, and a culture method using the same. In addition, KR Unexamined Patent Application Publication No. 10-2019-0118031 discloses a composition for promoting the differentiation of urine-derived multipotent stem cells containing dihydroxyflavone as an active ingredient in order to improve the ability to differentiate into various human constituent cells using induced multipotent stem cells, and a differentiation promoting method.

KR Patent No. 10-2016257 discloses a method of isolating urine-derived stem cells, which includes, in order to isolate urine-derived stem cells that can differentiate into several tissues and organs, like other somatic stem cells, collecting urine from a subject, collecting cells by centrifuging the urine, providing cells by centrifugation after washing the cells, and inoculating cells into a cell culture plate coated with a coating material.

KR Unexamined Patent Application Publication No. 10-2019-0003301 discloses a method of inducing the dedifferentiation of keratinocyte stem cells from urine cells by introducing reprogramming factors Bmi1 and dNP63a into urine-derived cells and a composition for promoting skin regeneration, which includes an induced reprogrammable keratinocyte stem cell-conditioned medium as an active ingredient.

In addition, KR Unexamined Patent Application Publication No. 10-2016-0050091 relates to a method of culturing urine progenitor cells from urine progenitor cells and a urine sample, and discloses a kit for isolating urine progenitor cells using a selective cell culture medium and a specific cell marker based on morphological characteristics.

However, the above patent documents do not disclose that urine samples or urine-derived cells left for a long time have epithelial cell characteristics and mesenchymal cell characteristics.

### DISCLOSURE

### Technical Problem

The present invention is directed to providing a medium composition for isolating multipotent cells from urine and massively proliferating the cells and a method of massively proliferating urine-derived multipotent cells using the same.

### Technical Solution

Accordingly, the present inventors confirmed that, when collecting a urine sample, by containing urine in a tube containing a precipitate solution for urine collection according to the present invention, the pH of the urine may be maintained in a neutral state for a long time, and although the urine is left for a long time, urine cells can be effectively isolated from the urine contained in the tube. In addition, a medium composition that can massively proliferate the urine cells isolated from the urine into multipotent cells having both epithelial cell and mesenchymal cell characteristics was specified, and the present invention was completed.

Therefore, the present invention relates to a medium composition for isolating urine-derived multipotent cells and massively proliferating the cells, and a method of massively proliferating urine-derived multipotent cells using the same.

Hereinafter, the composition of the present invention will be described in detail.

The present invention provides a method of massively proliferating urine-derived multipotent cells, which includes:
(a) isolating multipotent cells in urine by centrifuging the urine in a tube containing a precipitate solution for urine collection, prepared by adding serum; a growth factor; an antibiotic; insulin; an estrogen steroid hormone; a corticosteroid-based compound; a cytokine; a plasma-derived component; and the extracellular matrix to a basal medium for cell culture, and
(b) culturing the isolated multipotent cells in a medium composition for massively proliferating urine-derived multipotent cells, prepared by adding a plasma-derived component; a growth factor; insulin, an estrogen steroid hormone; a corticosteroid-based compound; and a cytokine are added to a basal medium for cell culture.

First, in (a), the precipitate solution for urine collection is prepared by adding serum; a growth factor; an antibiotic; insulin; an estrogen steroid hormone; a corticosteroid-based compound; a cytokine; a plasma-derived component; and the extracellular matrix to a basal medium for cell culture.

In the present invention, the basal medium for cell culture may be selected from the group consisting of Basal Media Eagle (BME), Dulbecco’s Modified Eagle’s Media (DMEM), RPMI medium, a 1:1 mixture of Dulbecco’s Modified Eagle’s Medium/F-12 Nutrient Mixture Ham (DMEM/F-12), RPMI1640, a 3:1 mixture of DMEM/F-12, Williams’ Medium E, Waymouth MB 752/1, Minimum Essential Media (MEM), Alpha Modification of Minimum Essential Media Eagle (α-MEM), Media 199 (M 199), MCDB medium, modified McCoy’s 5A medium, Leibovitz’s L-15 medium, Iscove’s Modified Dulbecco’s Media (IMDM), Fischer medium, CMRL 1066 medium, neuronal cell in brain (N2-medium), neuronal cell in brain (NB-medium) and Keratinocyte basal Medium (KBM), but the present invention is not limited thereto. As the medium, any medium having a composition that provides conditions for culturing urine-derived cells may be used without limitation.

In one embodiment, the basal medium may be selected from the group consisting of DMEM, RPMI, and Waymouth MB 752/1.

The basal medium may be included in a precipitate solution for urine collection at a content of 100 to 1000 ml, but the present invention is not limited thereto. The content may be adjusted by an amount of the collected urine sample.

In the present invention, the serum may be one or more selected from the group consisting of fetal bovine serum, calf serum, rabbit serum, goat serum, mouse serum, horse serum, sheep serum, pig serum, chicken serum, and human serum, but the present invention is not limited thereto.

In one embodiment, the serum may be fetal bovine serum.

In the present invention, the basal medium may contain 1% to 15% serum.

In the present invention, a growth factor added to the basal medium may be one or more growth factors selected from the group consisting of an epidermal growth factor (EGF), a platelet-derived growth factor (PDGF), a vascular endothelial growth factor (VEGF), a fibroblast growth factor (FGF), an insulin-like growth factor (IGF), and a leukemia inhibitory factor (LIF), but the present invention is not limited thereto.

In one embodiment, the growth factor may consist of an epidermal growth factor (EGF), a fibroblast growth factor (FGF) and a leukemia inhibitory factor (LIF).

The growth factor may be included in the basal medium to have a final concentration of 0.1 ng/ml to 1 µg/ml, for example, 0.5 ng/ml to 100 ng/ml, 0.5 ng/ml to 50 ng/ml, 1 ng/ml to 20 ng/ml, 5 ng/ml to 15 ng/ml, or 8 to 12 ng/ml. The total growth factors included in the basal medium may be contained at a concentration of 1 ng/ml to 50 ng/ml in the basal medium.

In the present invention, the antibiotic may include penicillin and/or streptomycin, but the present invention is not limited thereto.

The antibiotic may be included in the basal medium to have a final concentration of 0.1 µg/ml to 1 ml/ml, for example, 0.5 µg/ml to 500 µg/ml, or 1 µg/ml to 100 µg/ml.

In the present invention, the insulin may be included in the basal medium to have a final concentration of 100 ng/ml to 1 mg/ml, for example, 300 ng/ml to 500 µg/ml, 400 ng/ml to 100 µg/ml, 500 ng/ml to 50 µg/ml, 600 ng/ml to 10 µg/ml, 700 ng/ml to 5 µg/ml, 800 ng/ml to 3 µg/ml, or 900 ng/ml to 2 µg/ml.

In the present invention, the estrogen steroid hormone may be estradiol, but the present invention is not limited thereto. In the present invention, the estrogen steroid hormone may be included in the basal medium to have a final concentration of 1 to 30 nM, for example, 5 to 20 nM, or 8 to 15 nM.

In addition, the corticosteroid-based compound may be one or more selected from the group consisting of corticosterone, dexamethasone and hydrocortisone, but the present invention is not limited thereto. In one embodiment, and the corticosteroid-based compound may be hydrocortisone. In the present invention, the corticosteroid-based compound may be included in the basal medium to have a final concentration of 1 ng/ml to 1 µg/ml, for example, 1.5 to 500 ng/ml, 2 to 300 ng/ml, 3 to 100 ng/ml, 4 to 50 ng/ml, 5 to 30 ng/ml, 8 to 20 ng/ml, or 9 to 15 ng/ml.

In addition, the cytokine may be one or more selected from the group consisting of an interleukin, interferon-gamma and TGF-β, but the present invention is not limited thereto. In one embodiment, the cytokine may be interleukin-2 (IL-2). In the present invention, the cytokine may be included in the basal medium to have a final concentration of 0.1 to 100 ng/ml, for example, 0.5 to 50 ng/ml, 1 to 40 ng/ml, 3 to 30 ng/ml, 5 to 20 ng/ml, or 8 to 15 ng/ml.

In addition, the plasma-derived component may be selected from the group consisting of albumin, hemoglobin, and transferrin, but the present invention is not limited thereto. In one embodiment, the plasma-derived component may be albumin and hemoglobin. In the present embodiment, the albumin may be included in the basal medium to have a final concentration of 1 ng/ml to 1 µg/ml, for example, 1.5 to 500 ng/ml, 2 to 300 ng/ml, 3 to 100 ng/ml, 4 to 50 ng/ml, 5 to 30 ng/ml, 6 to 20 ng/ml, or 8 to 15 ng/ml, and the hemoglobin may be included in the basal medium to have a final concentration of 1 µg/ml to 1 mg/ml, for example, 1.5 to 500 µg/ml, 2 to 300 µg/ml, 3 to 100 µg/ml, 4 to 50 µg/ml, 5 to 30 µg/ml, 6 to 20 µg/ml, or 8 to 15 µg/ml.

In addition, the extracellular matrix may be one or more selected from the group consisting of collagen, laminin, fibronectin, gelatin, elastin and hyaluronic acid, but the present invention is not limited thereto. In the present invention, the collagen may be included in the basal medium to have a final concentration of 1 µg/ml to 1 mg/ml, for example, 1.5 to 500 µg/ml, 2 to 300 µg/ml, 3 to 100 µg/ml, 4 to 50 µg/ml, 5 to 30 µg/ml, 6 to 20 µg/ml, or 8 to 15 µg/ml; the laminin may be included in the basal medium to have a final concentration of 1 µg/ml to 1 mg/ml, for example, 1.5 to 500 µg/ml, 2 to 300 µg/ml, 3 to 100 µg/ml, 4 to 50 µg/ml, 5 to 30 µg/ml, 6 to 20 µg/ml, or 8 to 15 µg/ml; the fibronectin may be included in the basal medium to have a final concentration of 1 µg/ml to 1 mg/ml, for example, 1.5 to 500 µg/ml, 2 to 300 µg/ml, 3 to 100 µg/ml, 4 to 50 µg/ml, 5 to 30 µg/ml, 6 to 20 µg/ml, or 8 to 15 µg/ml; the gelatin may be included in the basal medium to have a final concentration of 1 µg/ml to 1 mg/ml, for example, 10 to 500 µg/ml, 30 to 300 µg/ml, 50 to 200 µg/ml, or 80 to 150 µg/ml; the hyaluronic acid may be included in the basal medium to have a final concentration of 1 µg/ml to 1 mg/ml, for example, 1.5 to 500 µg/ml, 2 to 300 µg/ml, 3 to 100 µg/ml, 4 to 50 µg/ml, 5 to 30 µg/ml, 6 to 20 µg/ml, or 8 to 15 µg/ml. In one embodiment, the extracellular matrix may consist of collagen, gelatin, and hyaluronic acid.

Since there are only a small amount of urine cells in urine, when the urine is collected in a tube containing the precipitate solution for urine collection according to the present invention, since the pH of the urine may be maintained in the range of 7 to 8, the survival of the urine cells contained in the urine may be extended, so that the urine cells may be efficiently isolated from a urine sample in the tube left for a long time. That is, a solution containing the precipitate solution for urine collection and the urine may have a pH of 7 to 8, for example, 7.2 to 7.6, or 7.3 to 7.5 when left at room temperature (20 to 30° C.) for 0 to 96 hours. Therefore, multipotent cells in the urine, isolated in (a), may be isolated from the urine after being left for 0 to 96 hours in the precipitate solution for urine collection.

In (a), the urine and the precipitate solution for urine collection may be included at a content ratio of 1:100 to 1:0.01, for example, 1:50 to 1:1, or 1:30 to 1:10. At the above-mentioned content ratio, the effect of maintaining the survival of urine cells may be maximized even in the urine sample that has been left for a long time.

In addition, in (a), the isolation of multipotent cells in the urine from the urine collected in the tube containing the precipitate solution for urine collection may be performed by performing centrifugation at room temperature (20 to 30° C.) and 1500 to 2500 rpm for 3 to 10 minutes.

Next, the urine-derived multipotent cells isolated in (a) is subjected to (b) primary culture in a medium composition for mass proliferation.

This step is to perform culture in a culture plate by adding a medium composition for mass proliferation to a precipitate obtained after centrifugation and pipetting several times to allow the precipitated state to become a suspended state in order to perform the primary culture of the multipotent cells in the urine, which have been previously isolated.

The medium composition for mass proliferation includes a plasma-derived component, a growth factor, and a cytokine. In the medium composition, the plasma-derived component, the growth factor, and the details of the plasma-derived component, the growth factor, and the cytokine, which have been described in (a), may be applied as they are.

The method according to the present invention may further include, after (b), (c) proliferating the culture cells by subculture. Here, the medium composition used in the primary culture may be used as a medium for the subculture.

This step is a step of transferring the primarily cultured cells to a clean bench, isolating the cells as a single cell using a trypsin-EDTA solution, and culturing a plurality of cells on a plate for mass proliferation. Here, cell proliferation may be confirmed by measuring the number of viable cells using a hematocytometer.

In the present invention, the term “subculture” used herein refers to a method of continuously culturing cells in passages by exchanging a culture medium with a fresh one after transferring some of the cells to a new culture container periodically to continuously culture the cells in a healthy state for a long time. It is used as a method to increase the number of healthy cells when the number of cells in a culture container with limited space increases, because after a certain period of time, proliferation nutrients are consumed or contaminants are accumulated, causing the cells to die naturally. In general, exchanging the medium (or culture container) with a fresh one once or splitting cell groups is referred to as 1 passage.

In the present invention, the subculture may be performed once or more, for example, four times or more, or seven times or more. According to the subculture, multipotent cells may be acquired with a substantially high purity (approximately 95% or more).

In (b) and (c), cell culture may be performed under conditions of 1 to 5% CO₂ and 30 to 40° C. for 5 to 20 days.

After (c), isolating epithelial cells, mesenchymal cells and hematopoietic stem cells from the massively-proliferated urine-derived multipotent cells may be further included.

The urine-derived multipotent cells massively proliferated by the method according to the present invention may have the characteristics of two or more cells selected from the group consisting of epithelial cells, mesenchymal cells and hematopoietic stem cells. That is, the urine-derived multipotent cells according to the present invention may express one or more of the epithelial cell marker cytokeratin 18 (CK18), an epithelial stem cell marker CD24, mesenchymal cell markers fibronectin and vimentin, and hematopoietic stem cell marker CD133, and epithelial cells, epithelial stem cells, mesenchymal cells and hematopoietic stem cells may be isolated and cultured according to the expression of the marker.

In addition, the present invention provides a medium composition for massively proliferating urine-derived multipotent cells, to which a plasma-derived component; a growth factor; insulin; an estrogen steroid hormone; a corticosteroid-based compound; and a cytokine are added to a basal medium for cell culture.

All the above details for a basal medium, a plasma-derived component, a growth factor, insulin, an estrogen steroid hormone, a corticosteroid-based compound and a cytokine, included in the medium composition for mass proliferation of the urine-derived multipotent cells, can be applied as they are.

In the present invention, the basal medium may be one selected from the group consisting of DMEM, RPMI and Waymouth MB 752/1.

In one embodiment, the basal medium may be DMEM.

In the present invention, the plasma-derived component may be one or more selected from the group consisting of albumin, hemoglobin and transferrin.

In one embodiment, the plasma-derived component may be albumin and/or hemoglobin.

Here, the plasma-derived component may include 1 to 100 ng/ml, for example, 3 to 50 ng/ml, or 5 to 20 ng/ml of albumin; and/or 0.01 to 100 µg/ml, for example, 0.1 to 50 µg/ml, 0.5 to 30 µg/ml, 1 to 20 µg/ml, or 5 to 15 µg/ml of hemoglobin, based on the total weight of the medium composition.

In the present invention, the growth factor may be selected from the group consisting of an epidermal growth factor (EGF), a fibroblast growth factor (FGF), and a leukemia inhibitory factor (LIF).

In the present invention, the estrogen steroid hormone may be estradiol. In one embodiment, the estrogen steroid hormone may be 17-B-estradiol.

In addition, the corticosteroid-based compound may be one or more selected from the group consisting of corticosterone, dexamethasone and hydrocortisone. For example, the corticosteroid-based compound may be hydrocortisone.

In addition, the cytokine may be one or more selected from the group consisting of interleukin, interferon-gamma, and TGF-β. For example, the cytokine may be interleukin-2 (IL-2).

Here, the medium composition may include 0.01 to 10 µg/ml of insulin and/or 1 to 20 ng/ml of hydrocortisone, based on the total weight of the medium composition.

According to the present invention, only when cultured in the above-described medium composition, the urine-derived cells may be massively proliferated as cells having the characteristics of two or more cells selected from the group consisting of epithelial cells, mesenchymal cells and hematopoietic stem cells.

Hereinafter, the advantages and features of the present invention and the methods of accomplishing the same will become apparent with reference to the detailed description of exemplary embodiments and the accompanying drawings. However, the present invention is not limited to the exemplary embodiments disclosed below, and may be embodied in many different forms. These exemplary embodiments are merely provided to complete the disclosure of the present invention and fully convey the scope of the present invention to those of ordinary skill in the art, and the present invention should be defined by only the accompanying claims.

### Advantageous Effects

According to a method of massively proliferating urine-derived multipotent cells and a medium composition for massively proliferating urine-derived multipotent cells according to the present invention, urine cells can be efficiently isolated from urine that has been left for a long time, and multipotent cells having the characteristics of epithelial cells, mesenchymal cells and hematopoietic stem cells can be prepared.

### DESCRIPTION OF DRAWINGS

FIG. 1 is a schematic diagram of a method of massively proliferating urine-derived multipotent cells using a precipitate solution for urine collection.

FIG. 2A shows a process of collecting urine in a tube, in which the left panel shows the result of leaving urine that has been collected in a tube containing a precipitate solution for urine collection for 72 hours, and the right panel shows the result of leaving urine that has been collected in a tube not containing a precipitate solution for urine collection for 72 hours, and FIG. 2B shows the precipitation result for the precipitate solution for urine collection and the urine cells by centrifugation of the collected urine.

FIG. 3 shows the process of primarily culturing the collected urine-derived cells in a culture plate, in which FIG. 3A shows the result of culturing the urine cells isolated from the urine that has been left for 72 hours in a tube not containing a precipitate solution for urine collection, and FIG. 3B shows the result of culturing the urine cells isolated from the urine that has been left for 72 hours in a tube containing a precipitate solution for urine collection.

FIG. 4 shows that the number of cells primarily cultured in a culture plate increases over time.

FIG. 5 shows the change in the number of viable cells that are proliferating after subculture.

FIG. 6 shows the result of observing the morphology of urine-derived cells cultured in a medium not containing a plasma-derived component, a growth factor and a cytokine.

FIG. 7 shows the result of observing the morphological characteristic of cells after the urine-derived cells of the present invention have been cultured for 3 or more passages, a breast cancer epithelial cell line MCF7, and adipose-derived cells, which are mesenchymal cells, are cultured for the same period and under the same culture conditions.

FIG. 8 shows the result of confirming whether an epithelial cell marker (CK18) and mesenchymal cell markers (fibronectin and vimentin) are expressed after the urine-derived cells of the present invention have been cultured for 3 or more passages, a breast cancer epithelial cell line MCF7, and adipose-derived cells, which are mesenchymal cells, are cultured for the same period and under the same culture conditions.

FIG. 9 is the result showing that the urine-derived cells of the present invention express an epithelial cell marker (CK18) and mesenchymal cell markers (fibronectin and vimentin) at the same time.

FIG. 10 is the result showing that, as the urine-derived cells of the present invention are subcultured, a hematopoietic stem cell marker (CD133) and an epithelial stem cell marker (CD24) are expressed in certain amounts.

FIG. 11 shows that the urine-derived cells of the present invention are isolated and cultured in a group expressing an epithelial stem cell marker (positive) and a group not expressing an epithelial stem cell marker (negative) using a flow cytometer (FACS). a: the expression of epithelial stem cell marker (CD24) in urine-derived cells; b: the expression of epithelial stem cell marker (CD24) after isolating epithelial stem cell marker (CD24)-negative (CD24-) and -positive (CD24+) cells; c: epithelial stem cell marker-negative (CD24-) and -positive (CD24+) cells cultured in culture plates after isolation.

FIG. 12 is the result showing that, after staining urine-derived cells with the gene staining reagent Hoechst 33342, which is used for hematopoietic stem cell analysis and isolation, there is a hematopoietic stem cell population (side population) of urine-derived cells through flow cytometry. a: urine-derived cells with various sizes; b: showing that the proportion of a hematopoietic stem cell population (side population) depends on a cell size.

### MODES OF THE INVENTION

Hereinafter, the present invention will be described in detail with reference to examples of the present invention. The following examples only illustrate the present invention, but the scope of the present invention is not limited to the following examples.

### EXAMPLES

### Example 1: Isolation and Mass Proliferation of Urine-Derived Multipotent Cells

### 1) Collection of Urine Cells

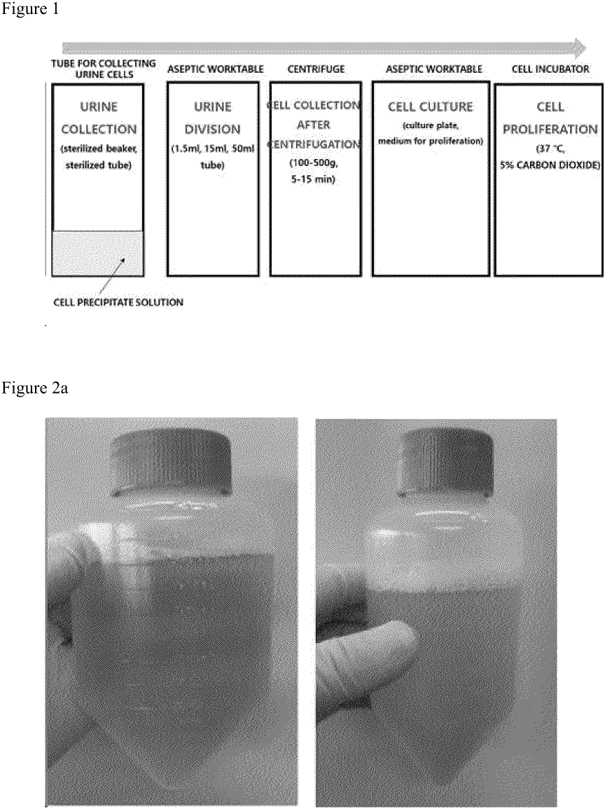

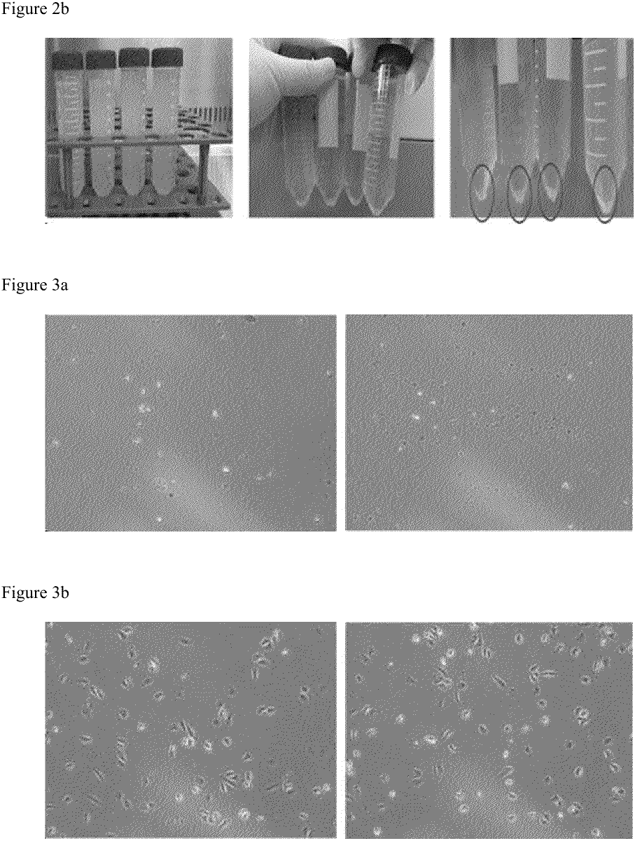

For urine collection, a precipitate solution for urine collection was prepared in DMEM, which is a basal medium, so that components for a composition shown in Table 1 below are contained in DMEM to have the corresponding final concentrations. The prepared precipitate solution for urine collection was put into a sterilized 250-ml tube, and then 200 ml of urine was collected (FIG. 2A). Here, 150 ml of the urine in the tube was isolated and left at room temperature for 72 hours. To compare a cell yield according to whether or not to leave the urine, urine was collected in a sterilized 250-ml tube not containing a precipitate solution for urine collection and then left at room temperature for 72 hours. A precipitate solution in which urine cells were precipitated was obtained by transferring the urine collected in the tube to a clean bench and adding the urine into a 50-ml tube, and centrifuging the urine in a centrifuge at a room temperature and 2,000 rpm for 5 minutes (FIG. 2B).

TABLE 1

| | | |
|---|---|---|
| Component | | Final concentration |
| Growth factor | bFGF | 10 ng/ml |
| | hEGF | 10 ng/ml |
| | LIF | 10 ng/ml |
| Insulin | Insulin | 1 µg/ml |
| Cytokine | IL-2 | 10 ng/ml |
| Corticosteroid-based compound | Hydrocortisone | 10 ng/ml |
| Estrogen steroid hormone | 17-B-estradiol | 10 nM |
| Plasma-derived component | Albumin | 10 ng/ml |
| | Hemoglobin | 10 µg/ml |
| Antibiotic | Penicillin or streptomycin | 10 µl/ml |
| Serum | Fetal bovine serum | 10% |
| Extracellular matrix | Collagen | 10 µg/ml |
| | Gelatin | 100 µg/ml |
| | Hyaluronic acid | 10 µg/ml |

### 2) Primary Culture of Acquired Urine Cells (7 Days)

The cells acquired from the tube containing the precipitate obtained in 1) and the tube that did not contain the precipitate were put into a medium for primary culture of the composition of Table 2 below, transferred to a 100-pi cell culture plate by pipetting, and cultured in a 5% CO₂, 37° C. incubator for 7 days (FIGS. 3A and 3B). According to the culture, it was confirmed that urine cells isolated from the urine left for 72 hours at room temperature in the tube not containing the precipitate solution for urine collection were not cultured and proliferated in an incubator (FIG. 3A), and urine cells isolated from the urine cells left for 72 hours in the tube containing the precipitate solution for urine collection were cultured and proliferated (FIG. 3B).

TABLE 2

Composition of medium for mass proliferation

| Component | | Final concentration |
|---|---|---|
| Growth factor | bFGF | 10 ng/ml |
| | hEGF | 10 ng/ml |
| | LIF | 10 ng/ml |
| Insulin | Insulin | 1 µg/ml |
| Cytokine | IL-2 | 10 ng/ml |
| Corticosteroid-based compound | Hydrocortisone | 10 ng/ml |
| Estrogen steroid hormone | 17-B-estradiol | 10 nM |
| Plasma-derived component | Albumin | 10 ng/ml |
| | Hemoglobin | 10 µg/ml |
| Antibiotic | Penicillin or streptomycin | 10 µl/ml |
| Serum | Fetal bovine serum | 10% |

### 3) Primary Culture of Acquired Urine Cells (14 Days)

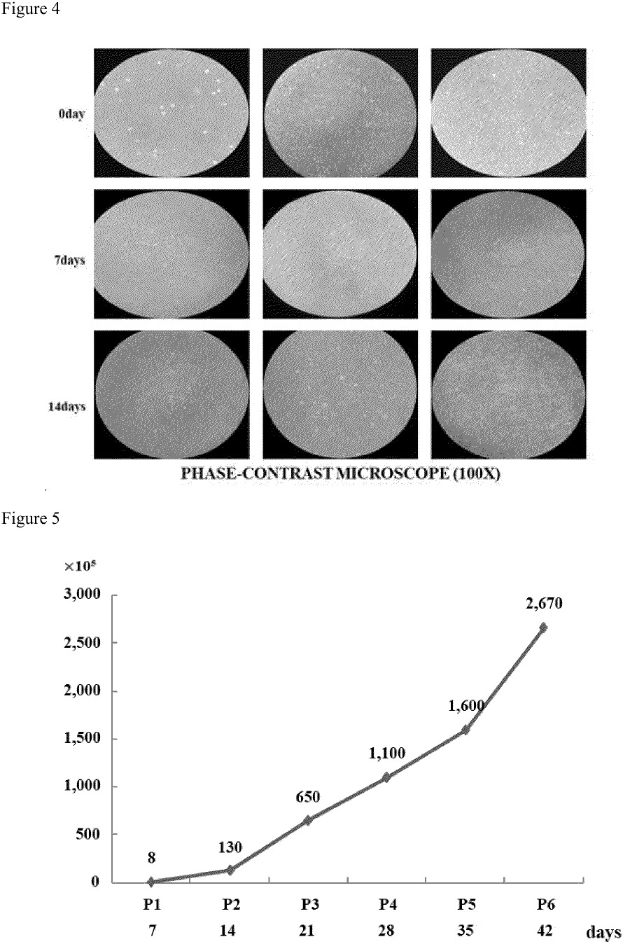

Urine cells acquired from urine were primarily cultured in the same manner as above except that the cells in 2) were cultured for 14 days (FIG. 4).

### 4) Mass Proliferation of Cells

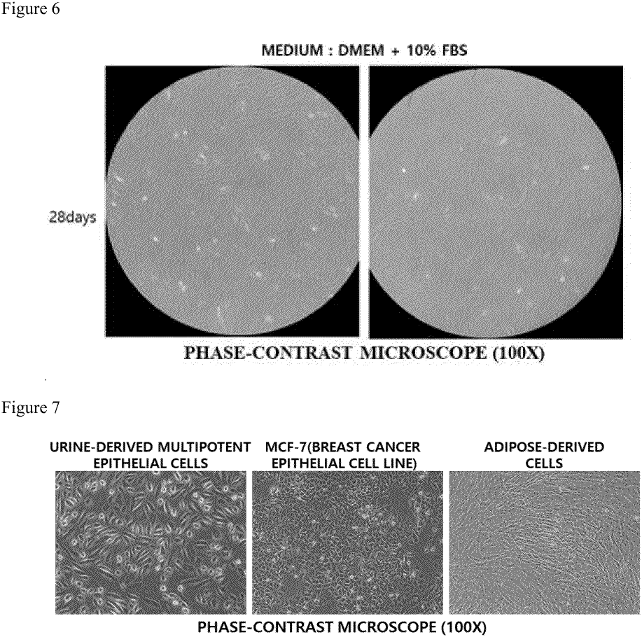

In order to massively proliferate the primarily cultured cells in 2) and 3), the primarily cultured cells were transferred to a clean bench, and cell states were observed through a microscope. Afterward, the cells were washed three times with PBS as a washing solution, and a trypsin-EDTA solution was added to a culture plate and left in an incubator for 10 minutes. After taking out the plate from the incubator and inactivating the trypsin-EDTA action using a cell culture medium, cells were collected, transferred to a tube and then centrifuged. During this process, to confirm cell proliferation, a part of the cell culture was taken, and then the number of viable cells was measured at each subculture. The subculture was performed every 7 days, and the number of viable cells measured at each subculture was compared with the initial number of viable cells just before the first suspension. As a result, it was confirmed that the number of viable cells increased at a constant rate at each subculture (FIG. 5). In addition, the morphology of cells was observed when cells were cultured in a medium that did not contain a “plasma-derived component, a growth factor and a cytokine,” which were contained in a medium for the mass proliferation of urine-derived multipotent cells according to the present invention (FIG. 6). As a result, the urine-derived cells cultured in the medium that did not include the “plasma-derived component, the growth factor and the cytokine” show the characteristics of typically aged cells that increase in size and grow slower as the culture period elapses, and therefore, when urine-derived cells were cultured in the medium for mass proliferation of the urine-derived cells according to the present invention, a mass proliferation effect, that is, an increased number of cells, can be confirmed.

### Example 2: Cell Characterization

### 1) Morphological Characterization

In order to confirm the characteristics of the cells cultured and proliferated in Example 1, breast cancer cell line MCF7, which is a representative cell line having an epithelial cell characteristics, adipose-derived cells, which are mesenchymal cells, and the urine-derived cells of Example 1, which were subcultured three or more times, were cultured using the same medium for the same period of time (72 hours) on 18-mm coverslips coated with 10 µg/ml of type IV collagen in 24-well plates. Four hours after culture, all cells were attached, and 72 hours after culture, their morphological characteristics were observed. As a result, it was seen that the urine-derived cells, compared to the adipose cells, which are mesenchymal cells, have a smaller size and overall oval shapes. In addition, the epithelial cancer cells MCF7 showed small and layered cells, whereas the urine-derived cells of the present invention showed the characteristics of larger and oval-shaped squamous cells, compared to the MCF7 cells (FIG. 7).

### 2) Confirmation by Immunostaining

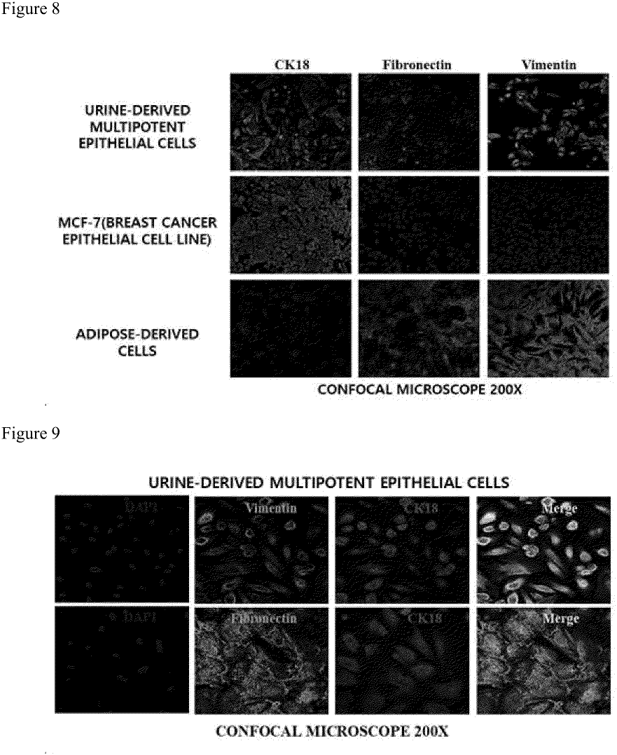

In order to confirm the characteristics of the cells cultured and proliferated in Example 1, the cells were fixed with paraformaldehyde, and subjected to immunocytochemistry. Cytokeratin 18 (CK18) was used as an epithelial cell marker, and fibronectin and vimentin were used as mesenchymal cell markers. Immunocytochemistry was performed as follows: the cultured cells on the 18-mm cover slip were washed with PBS, and fixed with a 4% formaldehyde solution at room temperature for 15 minutes. After washing with PBS three times, 0.5% Triton X-100 was permeated into the cells at room temperature for 15 minutes, and then the cells were washed with PBS three times. After blocking with 10% normal goat serum at room temperature for 1 hour, the cells were washed several times with PBS. The cells were left with a primary antibody for 1 hour. The cells were washed several times with PBS, left with a secondary antibody for 1 hour, and then washed three times with PBS. Afterward, the immunostained cells were stored in a VECTASHIELD mounting medium, and whether or not the markers were expressed was confirmed using a confocal microscope. As an observation result, the adipose-derived cells, which are mesenchymal cells, expressed the mesenchymal cell markers such as fibronectin and vimentin, the epithelial cells MCF7 did not express fibronectin and vimentin but expressed only the epithelial cell marker CK18 (FIG. 8). On the other hand, it was confirmed that the urine-derived cells according to the present invention express all of the mesenchymal cell markers such as fibronectin and vimentin, and the epithelial cell marker CK18 (FIG. 9). From the above result, it was able to be confirmed that the urine-derived cells according to the present invention have the characteristics of multipotent cells.

### 3) Confirmation of Multipotency

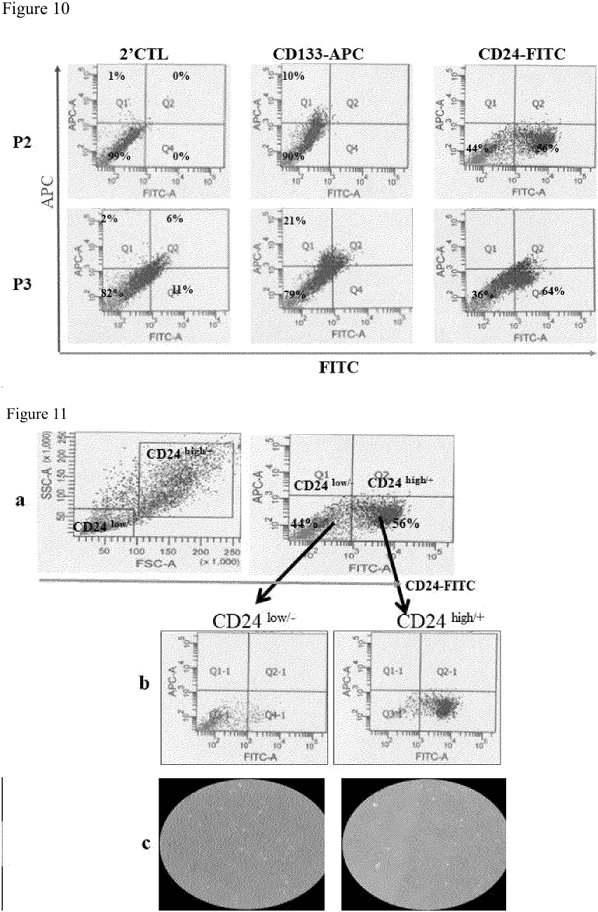

In order to confirm the multipotency of the urine-derived cells cultured and proliferated in Example 1, the expression of the hematopoietic stem cell marker CD133 and the epithelial stem cell marker CD24 was confirmed by a flow cytometer. The cells collected for flow cytometry were washed three times with PBS without fixing. The cells were blocked with 10% normal goat serum at room temperature for 1 hour, and washed several times with PBS. The cells were left with a secondary antibody-binding primary antibody for 1 hour at 4° C. without light. After washing three times with PBS, phenol red-free 10% FBS was added to confirm expression through flow cytometry (FIG. 10).

As a result of the observation, it was able to be confirmed that, even when the cells are subcultured, the expression pattern of a hematopoietic stem cell marker and an epithelial cell marker is increased or maintained (FIG. 10).

Based on the above result, it was able to be confirmed that the urine-derived cells according to the present invention have the characteristics of mesenchymal cells and hematopoietic stem cells as well as epithelial stem cells. In addition, it was able to be confirmed that positive cells show the morphology of epithelial cells, and negative cells show the morphology of mesenchymal cells by dividing these cells into the positive and negative cells based on the expression of an epithelial stem cell marker through flow cytometry (FIG. 11).

From the above results, it can be confirmed that epithelial stem cells and mesenchymal cells can be isolated from the urine-derived cells and proliferated.

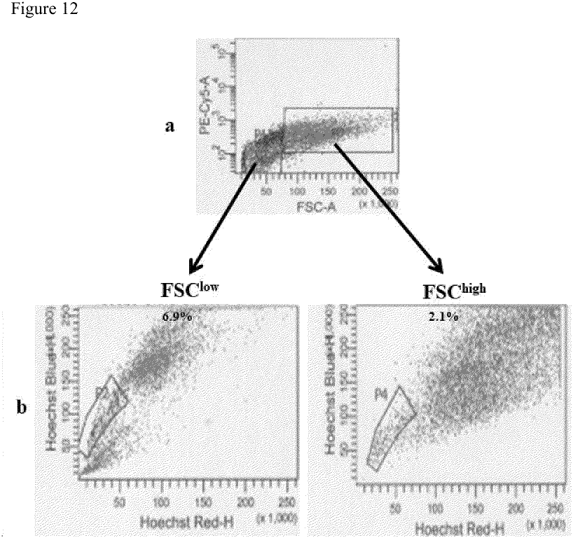

In addition, as a result of observing a side population using Hoechst 33342, which is a gene staining reagent for confirming hematopoietic stem cells, it was observed that there are side populations in all cell groups regardless of cell size (FIG. 12). This shows that hematopoietic stem cells can be isolated and cultured.

1. A method of massively proliferating urine-derived multipotent cells, comprising:
(a) isolating multipotent cells in urine by centrifuging the urine in a tube containing a precipitate solution for urine collection, prepared by adding serum; a growth factor; an antibiotic; insulin; an estrogen steroid hormone; a corticosteroid-based compound; a cytokine; a plasma-derived component; and the extracellular matrix to a basal medium for cell culture, and
(b) culturing the isolated multipotent cells in a medium composition for massively proliferating urine-derived multipotent cells, prepared by adding a plasma-derived component; a growth factor; insulin, an estrogen steroid hormone; a corticosteroid-based compound; and a cytokine are added to a basal medium for cell culture.

2. The method of claim 1, wherein, in (a), the basal medium for cell culture is one selected from the group consisting of DMEM, RPMI, and Waymouth MB 752/1.

3. The method of claim 1, wherein, in (a), the serum is one or more selected from the group consisting of fetal bovine serum, calf serum, rabbit serum, goat serum, mouse serum, horse serum, sheep serum, pig serum, chicken serum, and human serum.

4. The method of claim 1, wherein, in (a), the antibiotic is penicillin or streptomycin.

5. The method of claim 1, wherein the estrogen steroid hormone is estradiol, and
the corticosteroid-based compound is one or more selected from the group consisting of corticosterone, dexamethasone and hydrocortisone,
wherein the cytokine is one or more selected from the group consisting of an interleukin, interferon-gamma and TGF-β,
the plasma-derived component is one or more selected from the group consisting of albumin, hemoglobin and transferrin, and
the extracellular matrix is one or more selected from the group consisting of collagen, laminin, fibronectin, gelatin, elastin and hyaluronic acid.

6. The method of claim 1, wherein the growth factor is one or more selected from the group consisting of an epidermal growth factor (EGF), a platelet-derived growth factor (PDGF), a vascular endothelial growth factor (VEGF), a fibroblast growth factor (FGF), an insulin-like growth factor (IGF) and a leukemia inhibitory factor (LIF).

7. The method of claim 1, wherein, in (a), the multipotent cells in urine are isolated from the urine after being left for 0 to 96 hours in the tube containing the precipitate solution for urine collection.

8. The method of claim 1, wherein the urine collected in tube containing the precipitate solution for urine collection has a pH of 7 to 8 after 0 to 96 hours.

9. The method of claim 1, wherein, in (a), the precipitate solution for urine collection and the urine are comprised in a content ratio of 1:100 to 1:0.01.

10. The method of claim 1, further comprising, after (b), (c) proliferating the cultured cells by subculture.

11. The method of claim 1, wherein the urine-derived multipotent cells have two or more characteristics selected from the group consisting of epithelial cell characteristics, mesenchymal cell characteristics and hematopoietic stem cell characteristics.

12. A medium composition for the mass proliferation of urine-derived multipotent cells, comprising:
a basal medium; a plasma-derived component; a growth factor; insulin; an estrogen steroid hormone; a corticosteroid-based compound; and a cytokine.

13. The composition of claim 12, wherein the plasma-derived component is one or more selected from the group consisting of albumin, hemoglobin and transferrin,
the growth factor is one or more growth factors selected from the group consisting of an epidermal growth factor (EGF), a platelet-derived growth factor (PDGF), a vascular endothelial growth factor (VEGF), a fibroblast growth factor (FGF), an insulin-like growth factor (IGF), and a leukemia inhibitory factor (LIF),
the estrogen steroid hormone is estradiol,
the corticosteroid-based compound is one or more selected from the group consisting of corticosterone, dexamethasone and hydrocortisone, and
the cytokine is one or more selected from the group consisting of an interleukin, interferon-gamma and TGF-β.
